(19) United States
(12) Patent Application Publication
(10) Pub. No.: US 2019/0100726 A1
(43) Pub. Date: Apr. 4, 2019

(54) METHOD TO OBTAIN VEGETABLE FIBERS FROM THE ISOLATION AND CULTIVATION OF MERISTEMATIC CELLS

(71) Applicants: SUPER BAC - PROTEÇÃO AMBIENTAL S.A., Cotia (BR); UNIVERSIDAD DE ANTIOQUIA, Medellin (CO)

(72) Inventors: LUCIA ATEHORTUA GARCES, Medellín (CO); OSMAN DARIO FERNANDEZ BETIN, Medellín (CO); ANDRES DARIO LARA ESTRADA, Medellín (CO); CARLOS ENRIQUE MUSKLUS LOPEZ, Medellín (CO); ESTHER JULIA NARANJO GOMEZ, Medellín (CO); ORIANA PARRA ZULETA, Medellín (CO)

(73) Assignees: SUPER BAC - PROTEÇÃO AMBIENTAL S.A., Cotia (BR); UNIVERSIDAD DE ANTIOQUIA, Medellín (CO)

(21) Appl. No.: 16/151,670

(22) Filed: Oct. 4, 2018

(30) Foreign Application Priority Data
Oct. 4, 2017 (CO) NC2017/0010115

(51) Int. Cl. C12N 5/04

(52) U.S. Cl. CPC C12N 5/04

(57) ABSTRACT

A method to obtain vegetable fibers from vegetable specimen xylematic tissue meristematic cambial cells is provided. The method implies the identification and isolation of meristematic cambial cells from the xylematic tissue, their later cultivation and multiplication and the induction of fiber-like structures, in order to finally produce fibers. The process obtains vegetable fibers in laboratory conditions through isolated xylematic tissue cambial cells, offering an alternative to the production of fibers from plants.

### CROSS REFERENCE TO RELATED APPLICATIONS

This application claims priority under 35 U.S.C. § 119 to Colombian Application No. NC2017/0010115 filed on 4 Oct. 2017, the entire contents of which are incorporated herein by reference.

### BACKGROUND OF THE DISCLOSURE

### 1. Field of the Disclosure

This disclosure relates to biotechnological processes, particularly the in vitro propagation of vegetable xylematic tissue cambial cells and their conversion into products of industrial interest. Specifically, the disclosure relates to the isolation and cultivation of vegetable xylematic tissue meristematic cambial cells and the later acquisition of fibers from the cultivated cells.

### 2. Description of Related Art

The paper production sector is one of the industries with the highest environmental impact, since all stages of its process are potentially harmful. The first environmental impact begins with the consecution of the raw material. As the consumption of paper increases, one requires a larger quantity of trees to obtain the pulp, which is in many cases produced in monoculture, which in general reduces the productivity of the soil. Another source of trees for the paper industry are natural forests, which implies the deforestation and disturbance of ecosystems, significantly reducing the natural control over greenhouse effect gases.

An alternative to these issues could be the in vitro cultivation of vegetable cells and the laboratory production of fibers that can meet the needs of the paper industry and industries related to cellulose products. This would avoid deforestation issues and partially reduce the pollution produced by treatments made to transform log tissue into paper pulp. Likewise, it would contribute to the production of fibers regardless of external environmental and climate factors.

However, up to now there have been no reports in the established literature on a method to cultivate vegetable xylematic tissue meristematic cambial cells or on the production of fibers from said cells. The first limitation to the development of this type of technique consists of finding the proper cells to cultivate, which are able to transform into fibers.

Ogita, S, et al. discloses a means of cultivation that allows for the maintenance of plant cells in suspension to study the lignification process, especially in bamboo. See Ogita, S; Nomuro T., Kishimoto T, Kato E. 2012. A novel xylogenenic suspension culture model for exploring lignification in Phyllostachys bamboo. Plant Methods. 8:40. Likewise, Kumar et al. regenerated seedlings from the suspension of cambial cells from Dalbergia sissoo trees. The results showed that the shoots obtained were differentiated. See Kumar A, et. al. 1991. Morphogenesis response of cultured cells of cambial origin of a mature tree. Dalbergia sissoo Roxb. Plant Cell Rep. 9(12): 703-706.

Likewise, document U.S. Pat. No. 8,247,230 shows a method to isolate and obtain a homogenous cell line derived from the cambium that can divide itself. These cells are obtained from a tissue that contains cambium from an herbaceous plant, specifically storage roots of a herbaceous plant.

However, the state of the art does not include any report on the cultivation of woody specimen cells, not even of the conversion of these cells cultivated into fibers.

Added to that, an important restriction to the in vitro development of fibers is the paucity of knowledge existing in relation to the biology of the formation of fibers. Fibers are individual cells that belong both to the phloem and to the xylem, have an enlarged form, have a dense lignified secondary cell wall, and have sharp or pointy edges that result from a bend (knee) at the end of both edges of each cell. The identification of fibers in the first stages of differentiation is difficult and depends on the degree of development and characteristics of their position within the plant organ, and the presence of other distinctive characteristics, such as the longitudinal form and the width of the diameter of those typical of pro-cambial and cambial cells.

Likewise, the state of the art includes no report of biochemical, genetic or molecular markers of the premature development of fibers, due to the difficulty of seeing and obtaining fibers in their initiation processes, since when they are observed in premature differentiation stages still, the cell factors that specify the cellular destination of the fiber may have already ended their activity.

Therefore, there is a need in the state of the art for a method to obtain fibers from vegetable cells, identifying proper cells to be cultivated, proliferated and, later, allowing to obtain fibers that can be employed in different industries, such as the paper industry.

### BRIEF DESCRIPTION OF THE DISCLOSURE

This disclosure solves in an innovative manner the problems in the prior technique by providing a method to obtain fibers from vegetable specimen xylematic tissue meristematic cambial cells. The inventive method comprises:

(a) identifying and isolating vegetable meristematic cambial cells;
(b) multiplying isolated meristematic cambial cells;
(c) inducing the production of fiber-like structures from cultivated meristematic cambial cells;
(d) producing fibers from fiber-like structures.

In an advantageous manner, the method of the disclosure allows for the obtaining of in vitro fibers through biotechnological processes, the induction from these cells of fiber-like structures (which shall be named in this descriptive memorandum as “protofibers”) and, finally, the obtaining of fibers with industrial applications.

The disclosure also relates to a method for identifying and isolating meristematic cambial cells from the xylem tissue of plant species, which comprises staining the vegetable material with one or more staining reagents and performing tangential cuts at different plant heights, the identification by the microscope of the cells of interest and the obtaining of explants, which are introduced into a culture medium comprising an enzyme.

Another object of this disclosure is a method for the multiplication of meristematic cambial cells of plant xylem tissue, which holds the cells under suitable conditions for their subsequent differentiation. Such a method of multiplying meristematic cells comprises suspending these isolated meristematic cambial cells in a culture medium, creating a stable suspension and subculture the suspension in fresh culture medium.

The disclosure also relates to a method for obtaining fiber-like structures from cultured meristematic cambial cells of the cultured xylem tissue, which comprises suspending the meristematic cambial cells of the xylem tissue in a first culture medium, a subsequent suspension in a second culture medium, maintenance in the suspension for a first induction period, subsequent separation of the cells, and finally the addition of a second culture medium and maintenance of the suspension in agitation for a second induction period.

Likewise, the methods of this disclosure also include the transcriptomic analysis both of meristematic cambial cells and fiber-like structures (protofibers).

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows Cuts at 4 mm below the stem apex without stains. A: Cross-section 100 X. B: Tangential longitudinal cut 100 X.

FIG. 2 shows tangential Cuts of A: Xylem stained with pink Aniline and B: Xylem stained with blue Aniline 40 X.

FIG. 3 shows Cuts below the stem apex stained with an assisted lignin NBT marker. A: Cross-section at 100 X. B: Tangential cut at 100 X.

FIG. 4 shows the observation of cell feasibility. A: Clear field. B: Fluorescence filter.

FIG. 5 corresponds to A: Meristematic cells derived from the xylematic cambium: rounded, cored and vacuolated, clear yellow color, observed in a clear field, 40 X. B: Meristematic cells derived from the vascular cambium showing feasibility with FDA, 40 X.

FIG. 6 shows meristematic cells derived from the vascular xylematic cambium. A: Cells observed in DIC 40 X. B: Cells observed in polarized light 40 X. C-D: Cells stained with Calcoflour and showing the premature start of cell differentiation to give origin to a “Protofiber” 40 X.

FIG. 7 shows the start of the formation of fibers from meristematic cells derived from the vascular cambium. A: Clear field. B: DIC. C: Polarized light 40 X.

FIG. 8 shows the process of initiating a fiber-like structure, “Protofiber” originated from meristematic cells derived from the xylematic vascular cambium. A: Septal protofiber with well-defined chambers, with at least 4 cores (highlighted in the circles), emerging from meristematic cells undergoing cellular expansion. B: Active and feasible FDA protofiber 40 X.

FIG. 9 shows a Protofiber growing in a symplastic form. A: Stained with Calcoflour showing the presence of cellulose. B: DIC. C: Polarized light. In the latter, it is possible to observe the highly refractional edges, indicating the presence of crystalline cellulose and perhaps the reaccommodation of myofibrils for intrusive growth, 40 X.

FIG. 10 shows Protofibers with symplastic growth, with the start of an enlargement at the edges. A: DIC. B: Polarized light 40 X.

FIG. 11 shows Protofibers starting the intrusive growth process. A: Enlarged protofiber with walls undergoing an external thickening process. B: Enlarged protofiber showing the start of intrusive growth at both edges, 40 X.

FIG. 12 shows a Protofiber undergoing intrusion and thickening of the cell wall, probably in the process of deposition of the secondary lignin wall, DIC at 100 X.

FIG. 13 shows the differential centrifuging of the protofiber culture medium.

FIG. 14 shows the cultivation of E. grandis in greenhouse conditions.

FIG. 15 shows an example of the staining of E. grandis according to the inventive method.

### DETAILED DESCRIPTION

(a) This disclosure relates to a method to obtain vegetable fibers from xylem meristematic cambial cells, comprising the following steps: (a) identifying and isolating vegetable meristematic cambial cells from the xylem; (b) multiplying isolated meristematic cambial cells from the xylem; (c) inducing the production of fiber-like cells (protofibers) from cultivated meristematic cambial cells from the xylem; and (d) producing fibers from fiber-like cells (protofibers).

Thus the method of the disclosure allows for the obtaining of fibers in laboratory conditions from isolated xylem meristematic cambial cells, with said fibers being useful for industries requiring fibrous cellulosic raw materials, such as the paper industry.

In a preferred embodiment of the disclosure, step (a) of this method to obtain vegetable fibers occurs through the selective staining of cells. This staining allows for the exact and specific localization of xylem meristematic cambial cells proper for proliferation and their differentiation in fiber-like structures, “protofibers”. After the staining, the cells are isolated through histologic cuts at the parts of the plant where the proper meristematic cambial cells are located.

Once the cuts have been made, the cells are isolated from the tissue containing them and then taken to a first culture medium under in vitro conditions for multiplication according to step (b).

Preferably, this first culture medium comprises macronutrients, micronutrients, vitamins, antioxidants and phytohormones. In a preferred embodiment of the disclosure, phytohormones that are part of this first culture medium are selected from compounds similar to auxin, cytokines, gibberellins, ethylene, jasmonic acid and a mixture thereof.

According to an embodiment of the disclosure, macronutrients of the first culture medium of the disclosure comprise nitrogen, calcium, magnesium, potassium and phosphorus. Likewise, preferably the micronutrients comprise boron, cobalt, copper, iron, manganese, potassium, molybdenum and zinc. Preferably, the vitamins comprise B complex, polyalcohol and amino acids.

According to the method of the disclosure, after the multiplication of meristematic cells of step (b), the production of protofibers is induced, as per step (c), wherein the induction is made in a second culture medium.

Preferably, this step (c) of this disclosure is made in a second culture medium, comprising macronutrients, micronutrients, vitamins, antioxidants and phytohormones.

According to an embodiment of the disclosure, macronutrients of the second culture medium of the disclosure comprise nitrogen, calcium, magnesium, potassium and phosphorus. Likewise, preferably the micronutrients comprise boron, cobalt, copper, iron, manganese, potassium, molybdenum and zinc. Preferably, the vitamins comprise B complex, polyalcohol and amino acids.

Preferably, the phytohormones of the second culture medium are different from the phytohormones comprised in the first culture medium of step (b). In a preferred embodiment, the second culture medium comprises phytohormones selected from the group that consists of auxins, cytokines, gibberellins, brassinosteroids, ethylene and a mixture thereof.

Later, according to the disclosure, the protofibers of step (c) are transformed into proper vegetable fibers through the cultivation of protofibers and the induction of their lignification, for later apoptosis, thus originating true xylem fibers.

In a preferred embodiment of the disclosure, steps (b) and (c) are made in two stages, the first in a recipient subjected to stirring and the second in a bubble column-type bioreactor.

In another object of the disclosure, a new process is disclosed for the identification and isolation of xylematic tissue meristematic cambial cells, characterized in that it includes the steps of: (i) taking apex segments of stems from the vegetable material and submerging them in a solution of a first staining reagent; (ii) making tangential cuts at different heights of the plant; (iii) identifying stained cells under the microscope and identifying components of the xylematic tissue; (iv) taking apex segments of stems from the vegetable material and submerging them in a solution of a second staining reagent; (v) making cross-sections tangential cuts at different heights of the plant; (vi) analyzing stained cells under the microscope and identifying the height where the xylematic meristematic cambial cells of interest are located; (vii) making new tangential cuts of fine segments of the vegetable material at the height where the xylematic meristematic cambial cells of interest have been identified; (viii) introducing explants in a liquid culture medium containing an enzyme; and (ix) verifying the presence of free cells.

In a preferred embodiment of the disclosure, the first staining reagent of step (i) is a vital staining reagent. According to another preferred embodiment of the disclosure process, this vital staining reagent is selected from the group that consists of pink aniline and blue aniline.

Similarly, in a preferred embodiment of the disclosure, the staining process of step (i) is performed twice, once with pink aniline and another with blue aniline.

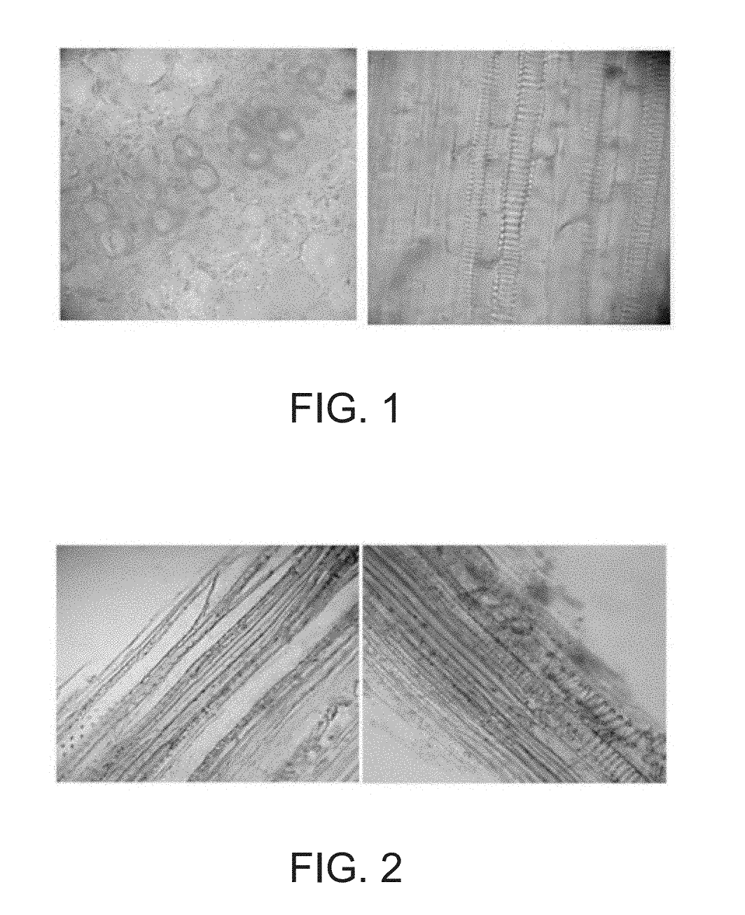

As shown in FIG. 1, the cuts without stains below the stem apex of a vegetable specimen allow for the distinction of different types of cells present, but not the meristematic cells that are able to differentiate into protofibers.

According to an object of the disclosure, the staining of step (i), both with aniline pink and with aniline blue, allows for the identification of xylem cells (FIG. 2). Thus, the cells that suffered apoptosis and that are not susceptible to multiplication are identified.

Later, according to the method of the disclosure, a second staining is performed in step (iv), allowing for the determination of xylematic meristematic cambial cells that are proper to be cultivated. According to a preferred embodiment of the disclosure, the second staining reagent is a chemical marker.

In a preferred embodiment of the disclosure method, the chemical marker of step (iv) allows for the synthesis of lignin. In an embodiment of the disclosure, the lignin marker is nitrotetrazolium blue (NBT).

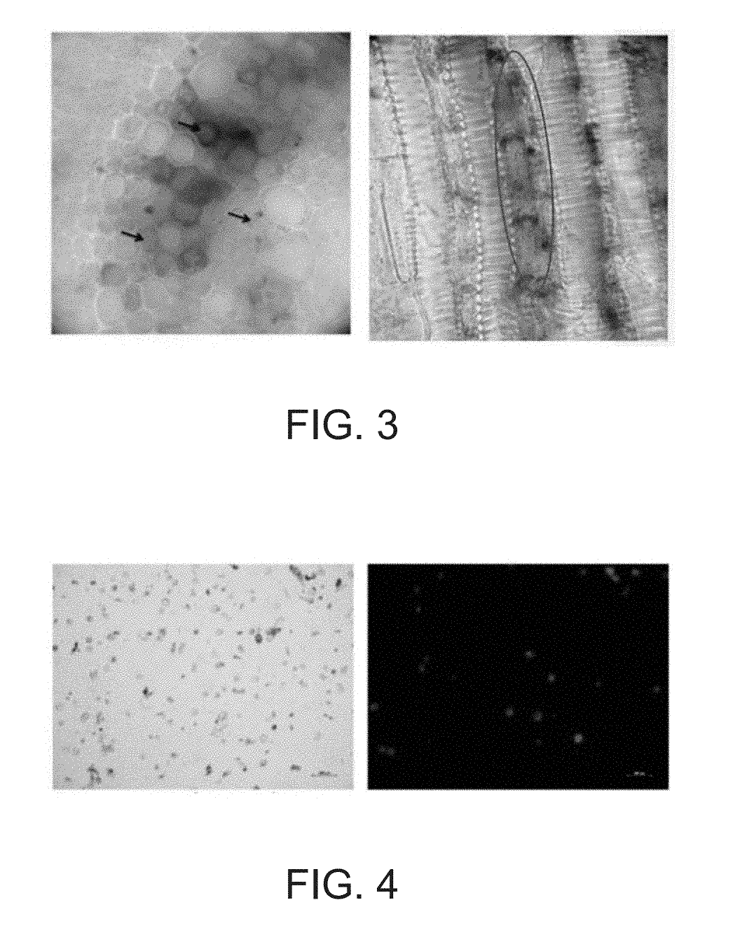

As shown in FIG. 3, according to a preferred embodiment of the disclosure after step (iv), transverse and tangential cross-sections are performed at different plant heights, according to step (v), and these are analyzed by means of the microscope, step (vi), where the meristematic cambial cells are identified. In FIG. 3, one may notice how the meristematic cambial cells stained in accordance with the disclosure process present a blue stain inside.

Once these cells are identified, according to step (vii) of the method of the disclosure, tangential cuts of thin segments of the plant material are made, obtaining explants, at the time the cambial cells of interest were identified under the microscope. According to a preferred embodiment of the disclosure, explants obtained as such are introduced in a culture medium that contains an enzyme.

Preferably, the enzyme contained in the liquid culture medium of step (viii) is selected from the group that comprises pectinases, cellulases, hemicelluloses and a cocktail thereof.

In an embodiment of the disclosure, the segments of apex stems are cut from plants that are between two months and one year and a half old after germination at the portions nearest to the apex region. These segments are submerged into the solution of the first staining reagent as per step (i) of the disclosure method. According to a preferred embodiment of the disclosure, the first staining reagent has a concentration between 20 mg/mL and 250 mg/mL.

According to this embodiment, the apex stem segments are submerged in the solution of the first staining reagent and left to rest for a first period of time. In a preferred embodiment of the disclosure, the first period of time is between 1 and 6 hours, preferably between 2 and 8 hours.

Preferably, after the staining of step (i), the cuts of step (ii) are made at heights of 1 mm to 8 cm below the stem apex.

On the other hand, in this embodiment of the disclosure, segments of apical stems are taken to step (iv) in a similar manner to step (i) and are submerged in a solution of a second staining reagent. Preferably, this second staining reagent has a concentration between 1 mg/mL and 10 mg/mL. Similarly to step (i) and (ii), the stems are left to rest for a second period of time, wherein this second period lasts between 1 and 8 hours, preferably between 2 and 5 hours, and the cuts of step (v) are made at heights of 1 mm to 8 cm below the stem apex.

According to this embodiment of the disclosure, in identifying the segments where the meristematic cells can be differentiated into protofibers, cuts are made according to step (vii) and the explants thus obtained are disinfected and treated under sterile conditions, then introduced in step (viii) in a liquid culture medium, which contains an enzyme. Preferably, this enzyme is at a concentration between 0.01% and 1.5% in this culture medium.

Preferably, the explants are left stirring for a third period of time. Preferably, this third period of time lasts between 4 and 30 days, but preferably between 5 and 8 days. Later, step (ix) is executed under sterile conditions. As seen in FIG. 4, in step (ix), one may verify the existence and feasibility of isolated cells.

According to an embodiment of the disclosure, steps (viii) and (ix) are practiced more than once.

Another object of this disclosure is a method for the multiplication of isolated meristematic cells. This method of the disclosure is characterized in that it comprises the steps of: (i) establishing a suspension of meristematic cells derived from the vascular xylematic cambium, free in a liquid culture medium; (ii) maintaining a stable suspension between 2 g/L and 20 g/L in dry weight of cells; and (iii) performing subcultures of the stable suspension of step (iii) adding between 1 g/L and 20 g/L in dry weight of cells in a fresh culture medium and maintaining subcultures under permanent stirring.

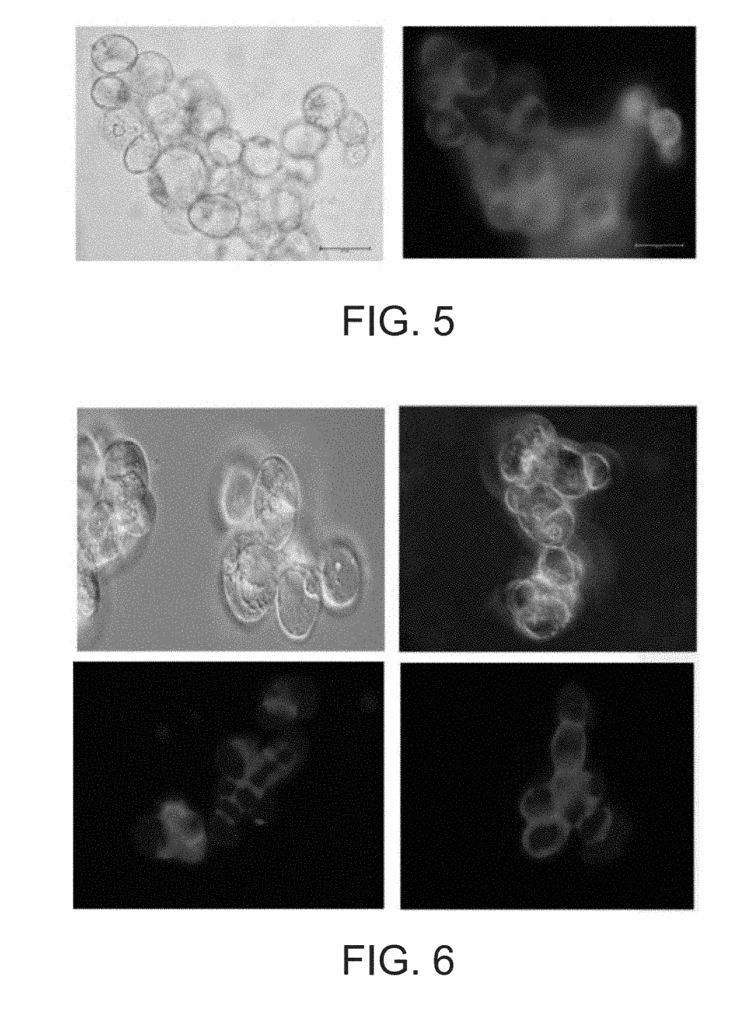

In a preferred embodiment of the disclosure, the method to multiply meristematic cells derived from the vascular cambium of the xylematic tissue, cultivated under luminous irradiation through a light-emitting diode system. FIG. 5 shows meristematic cells derived from the vascular cambium of the xylematic tissue, cultivated and feasible, as shown by their staining with Fluorescein Diacetate (FDA).

In another object, the disclosure shows a method to obtain fiber-like structures, “protofibers”, from meristematic cells derived from the vascular cambium, isolated and cultivated. This inventive method comprises: (i) suspending meristematic cambial cells in a first culture medium that comprises macronutrients, micronutrients, vitamins, antioxidants and phytohormones; (ii) taking the meristematic cambial cells from step (i) and suspending them again in a second culture medium that comprises macronutrients, micronutrients, vitamins, antioxidants and phytohormones; (iii) stirring the suspension for a first induction period; (iv) separating the cells from the culture medium and add the second fresh culture medium again; (v) stirring the suspension for a second induction period; wherein the second culture medium comprises phytohormones different from those of the first culture medium.

In an embodiment of the disclosure, the method to obtain protofibers of the disclosure is executed in two steps, the first being in a recipient subjected to stirring, and the second in a bubble column bioreactor.

According to one embodiment of the disclosure, the macronutrients of the first culture medium of the disclosure comprise nitrogen, calcium, magnesium, potassium and phosphorus. Likewise, preferably the micronutrients comprise boron, cobalt, copper, iron, manganese, potassium, molybdenum and zinc. Preferably, the vitamins comprise B-complexes, polyalcohol and amino acids.

Preferably, the phytohormones comprised in the first culture medium are selected from the group consisting of auxin, cytokines, gibberellins, ethylene, abscisic acid and mixtures thereof.

In one embodiment of the disclosure, the second culture medium comprises macronutrients including nitrogen, calcium, magnesium, potassium and phosphorus. Likewise, preferably the micronutrients comprise boron, cobalt, copper, iron, manganese, potassium, molybdenum and zinc. Preferably, the vitamins comprise B-complexes, polyalcohol and amino acids. On the other hand, said second culture medium comprises phytohormones which are selected from the group consisting of auxins, gibberellins, cytokines, abscisic acid and mixtures thereof.

According to one embodiment of the disclosure, to obtain protofibers from meristematic cells derived from the exchange, a suspension of such cells is carried out according to step (i) of between 3 and 20 g/L in dry weight and it is verified that the cell viability is greater than or equal to 75%.

For step (ii) in this embodiment of the disclosure, the suspension of step (i) is diluted to a concentration of 1.5 to 6.5 g/L in a second culture medium.

The suspension thus obtained is maintained in a stirred vessel for a first induction period according to step (iii), then separated therefrom according to step (iv) and suspended again in the second fresh culture medium to re-stir, according to step (v).

As indicated above, in one embodiment of the disclosure, the method of producing protofibers is performed in two steps, wherein the second step is performed in a bubble column-type bioreactor. In this embodiment, the reactor feed air stream is maintained between 0.25 L/min and 0.9 L/min.

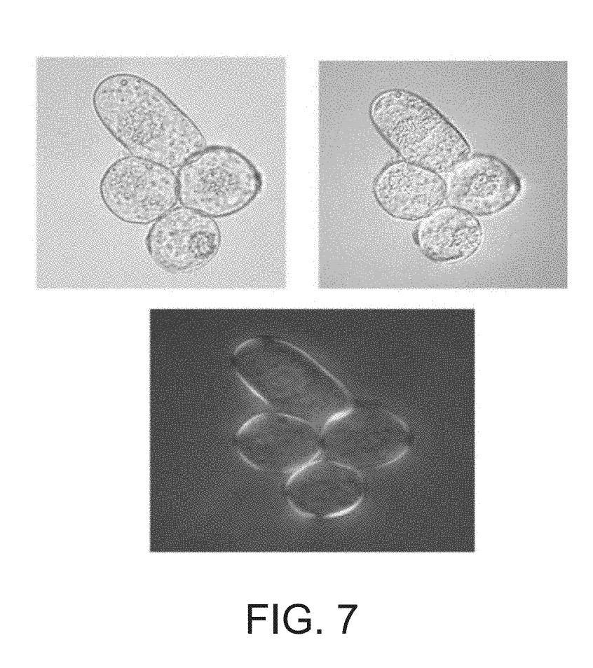

FIG. 6 shows the onset of meristematic cell differentiation in protofibers, likewise, FIG. 7 illustrates the onset of such protofiber formation from xylem meristematic cambial cells.

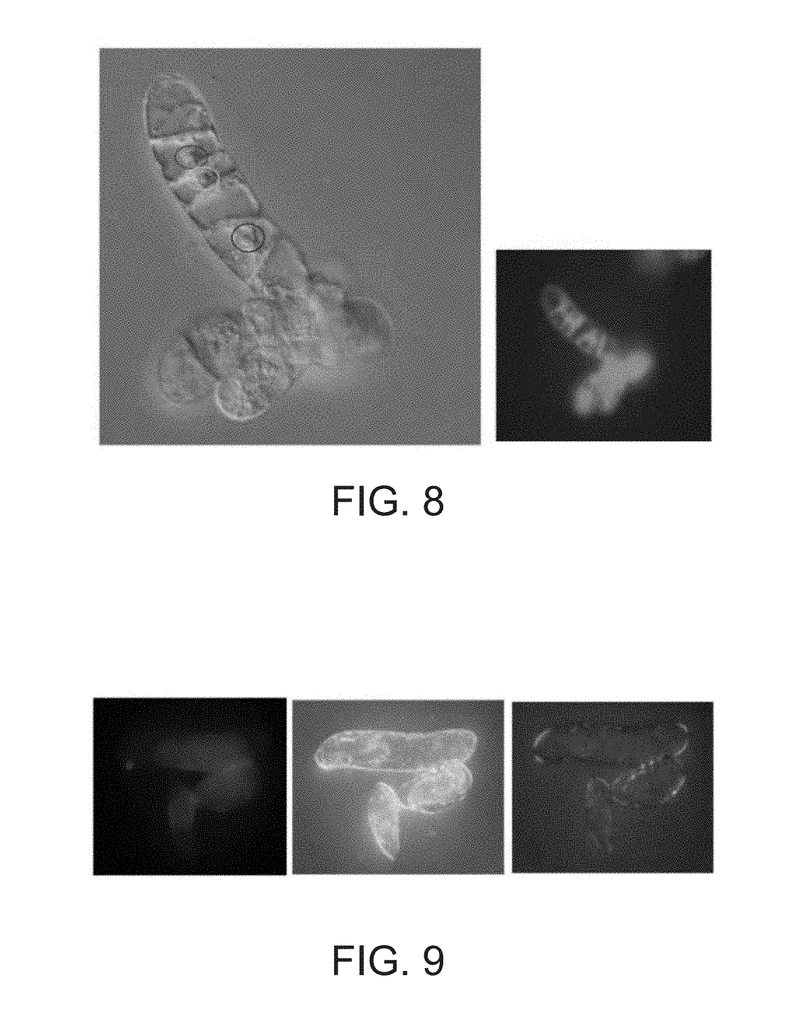

On the other hand, FIG. 8 shows an already septate Protofiber with at least four nuclei (marked in circles in the figure), which is emerging from the xylem meristematic cambial cells in processes of cellular expansion.

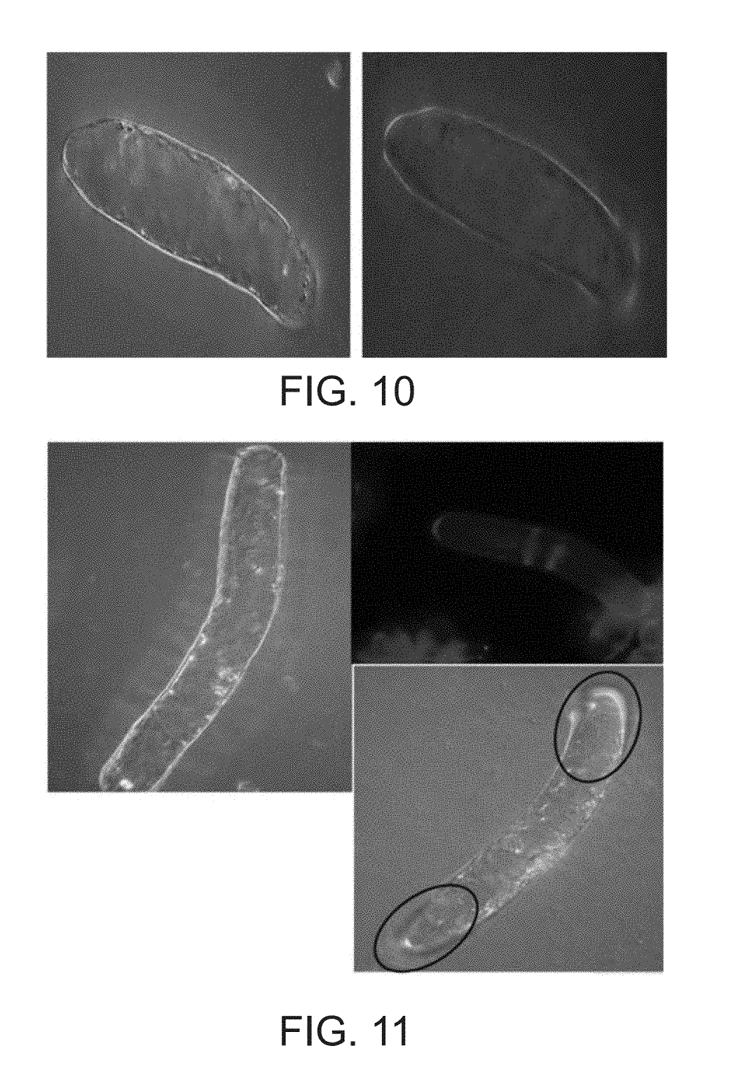

FIG. 9 shows protofibers growing in a symplastic form, where it is possible to evidence highly refractive edges. In turn, FIG. 10 shows how the protofibers generated and in symplastic growth initiate the enlargement of their edges.

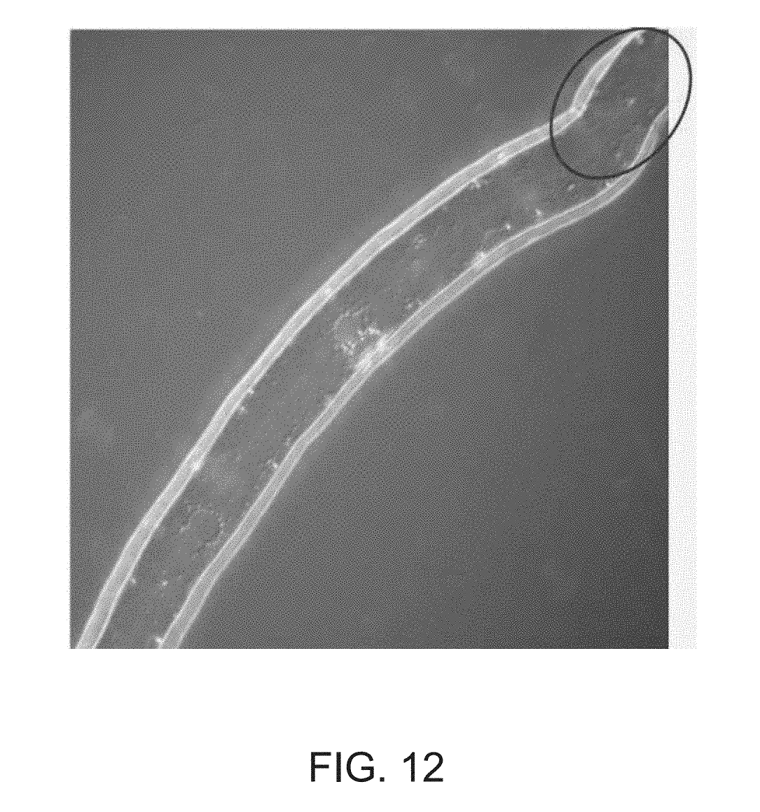

FIG. 11 shows how the protofibers generated begin the process of intrusive growth and external thickening process on their walls, as well as the beginning of intrusive growth at both edges. Similarly, FIG. 12 shows a Protofiber in the later process of cell wall intrusion and thickening.

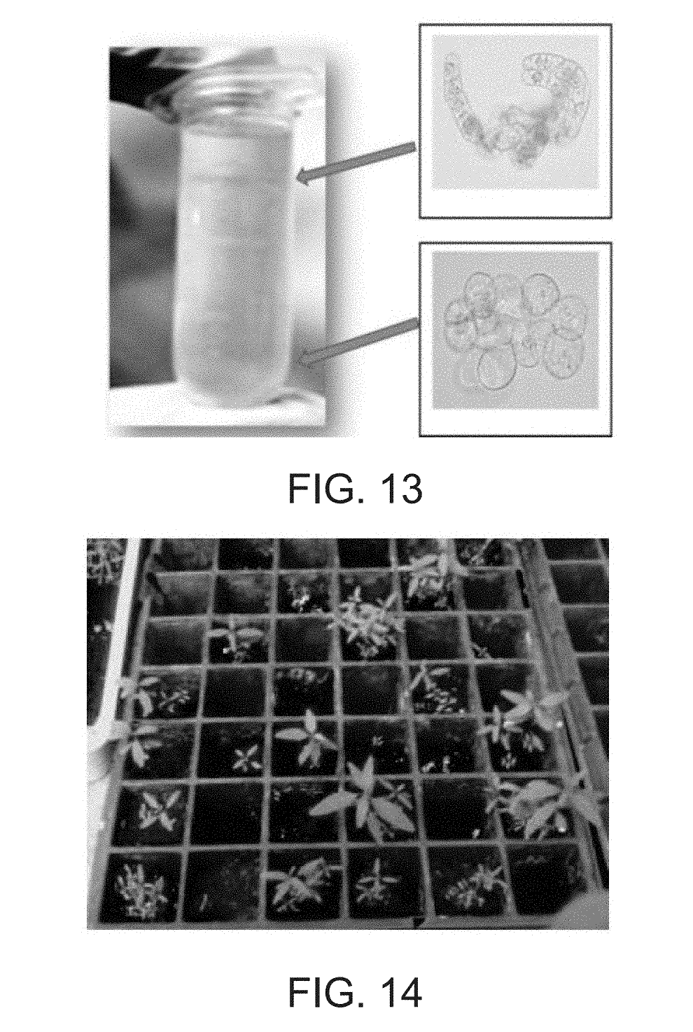

On the other hand, according to the method of obtaining protofibers of the disclosure, differential centrifugation of the suspension is performed during steps (iv) and (v) to separate the protofibers obtained from the meristematic cambial cells of the xylem tissue, yet without differentiating, which allows for the enrichening of the protofiber medium. FIG. 13 shows an example of differential centrifugation of the protofibers obtained by the method of the disclosure.

On the other hand, according to the present disclosure, the methods disclosed herein include an additional step in which the transcriptomic analysis of the isolated meristematic cambial cells and the obtained protofibers is performed. This analysis is generally performed with known RNA extraction and sequencing protocols.

### EXAMPLES

As reference plant material, Eucalyptus grandis (E. grandis), which is one of the most highly cultivated wood species in the world, was extracted for the quality of its wood and the presence of short fibers that makes it suitable as raw material in the industry paper.

E. grandis seeds were grown under greenhouse conditions to obtain seedlings from which meristematic cells were extracted. FIG. 14 shows germinated seedlings under greenhouse conditions.

### Example 1: Identification and Isolation of Meristematic Cells

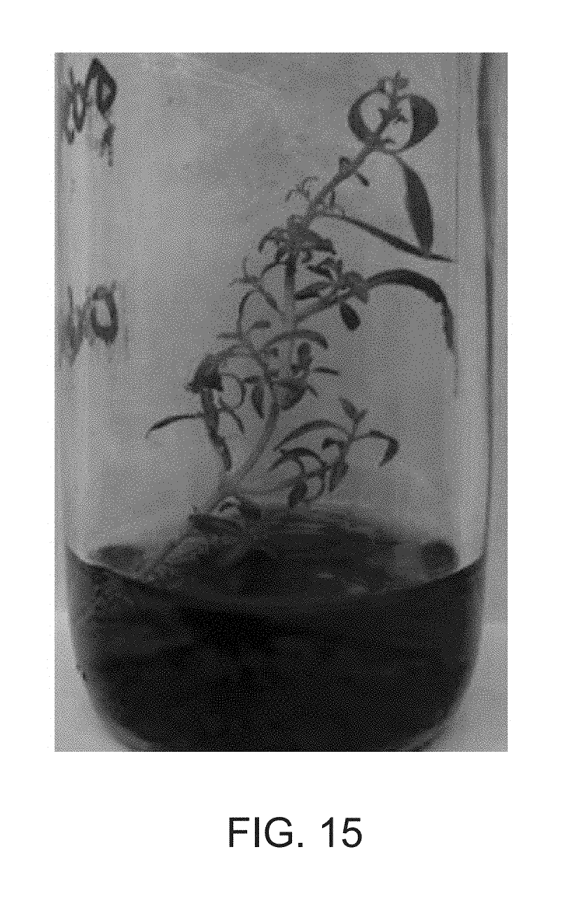

Aqueous solutions of Aniline Pink (around 50 mg/mL) and Aniline Blue (around 90 mg/mL) were prepared in aqueous solutions, in which segments of apical stems collected, approximately 8 cm long, were submerged (FIG. 15). The segments were left in the solution for 3-8 hours at room temperature. Subsequently, cuts were made at 4 mm, 1 cm, 2 cm and 4 cm below the stem apex, which were observed under the microscope.

On the other hand, an aqueous solution of Nitrotetrazolium Blue (N6876-SIGMA-ALDRICH) of about 3.0-5.0 g/L was prepared, segments of apical stems approximately 8 cm long were collected and left in the solution for 3-8 hours at room temperature. Subsequently, cuts were made at 4 mm, 1 cm, 2 cm and 4 cm below the stem apex, which were observed under the microscope.

After identifying the portions containing the exchangeable meristematic cells of the xylem tissue suitable for differentiation into protofibers, stem explants were obtained, which are introduced into sterile water and subsequently introduced into a solution with culture medium, supplemented with enzymes such as pectinase, cellulase, hemicellulose or mixtures thereof at a concentration between 3-10%.

The presence of free cells and their viability was analyzed by observation under the microscope.

### Example 2: Meristematic Cell Multiplication

A suspension of meristematic cells derived from the vascular exchange of the xylem tissue was performed in a culture medium under sterile conditions until it remained stable at a dry weight concentration of about 6 g/L.

Subcultures of this suspension were then performed inoculating between 2 and 10 g/L in a fresh culture medium.

### Example 3: Multiplication of Meristematic Cells Employing Luminous Radiation

Suspensions of about 6 g/L, exposed to perpendicular radiation by means of light-emitting diodes, are carried out at an intensity between 2 W/m² and 10 W/m². 24-hour light cycles are performed.

### Example 4: Protofiber Induction

Isolated meristematic cells with a viability percentage greater than 75% were suspended to provide a concentration of between 6 and 9 g/L in dry weight. The protofiber induction culture medium was introduced until a dilution of 1.5 and 4 g/L was obtained. The percentage of viability of the cells was analyzed and the concentration of the suspension remained stable.

The suspensions were taken to an orbital shaker, between 80 and 100 rpm. Subsequently, samples were taken to perform the feasibility percentage analysis, the concentration and the percentage of protofiber induction.

What is claimed is:

1. A method for obtaining fibers from plant meristematic cells, the method comprising the steps of:
(a) identifying and isolating vegetable meristematic cambial cells;
(b) multiplying the meristematic cambial cells;
(c) inducing production of fiber-like structures from the meristematic cambial cells; and
(d) producing fibers from the fiber-like structures.

2. The method of claim 1, wherein the step of identifying and isolating the meristematic cambial cells of step (a) is made by selective staining of cells.

3. The method of claim 1, wherein the step of identifying and isolating the meristematic cambial cells of step (a) is performed by histological sections at a point of a plant identified by staining.

4. The method of claim 1, wherein the step of multiplying the meristematic cambial cells of step (b) is performed by in vitro cell culture of the meristematic cambial cells in a first culture medium.

5. The method of claim 4, wherein the first culture medium comprises macronutrients, micronutrients, vitamins, antioxidants, and phytohormones.

6. The method of claim 5, wherein the phytohormones are selected from the group consisting of auxin, cytokines, gibberellins, ethylene, abscisic acid and mixtures thereof.

7. The method of claim 4, wherein the step of inducing production of the fiber-like structures of step (c) is performed in a second culture medium.

8. The method of claim 7, wherein the step of inducing production of the fiber-like structures of step (c) is performed in a first step and a second step, wherein the first step is in a stirred vessel and the second step is in a bubbling column type bioreactor.

9. The method of claim 7, wherein the second culture medium comprises macronutrients, micronutrients, vitamins, antioxidants, and phytohormones, wherein the phytohormones which are part of the second culture medium are different from the phytohormones present in the first culture medium.

10. The method of claim 9, wherein the phytohormones of the second culture medium are selected from the group consisting of auxins, gibberellins, cytokines, abscisic acid, and mixtures thereof.

11. A method for identifying and isolating exchange meristematic cells from xylem tissue, the method comprising the steps of:
(i) taking apex segments of stems from vegetable material and submerging the apex segments in a solution of a first staining reagent;
(ii) making tangential cuts at different heights of the stems;
(iii) identify, under a microscope, cells dyed by the first staining reagent and identify components of xylematic tissue;
(iv) taking the apex segments and submerging the apex segments in a solution of a second staining reagent;
(v) making cross-sections tangential cuts at different heights of the stems;
(vi) analyzing under the microscope cells dyed by the second staining reagent and identifying a height where meristematic cambial cells of interest are;
(vii) making new tangential cuts of fine segments of the stems at the height where the meristematic cambial cells of interest have been identified to obtain explants;
(viii) introducing the explants obtained in step (vii) into a liquid culture medium containing an enzyme; and
(ix) verifying the presence of free cells,
wherein the enzyme contained in the liquid culture medium of step (viii) is selected from the group consisting of pectinases, cellulases, hemicellulases, and mixtures thereof.

12. The method of claim 11, wherein the first staining reagent of step (i) is a vital staining reagent.

13. The method of claim 12, wherein the vital staining reagent is pink aniline and/or blue aniline.

14. The method of claim 11, wherein the second staining reagent is a chemical marker.

15. The method of claim 15, wherein the chemical marker allows the determination of lignin synthesis.

16. The method of claim 15, wherein the chemical marker is nitrotetrazolium.

17. A method for multiplying isolated meristematic cambial cells, the method comprising the steps of:
(i) establishing a suspension of free meristematic cambial cells in a liquid culture medium;
(ii) maintaining a stable suspension of between 2 g/L to 15 g/L in dry weight of cells; and
(iii) performing subcultures of the stable suspension of step (iii) by adding between 1 g/L and 15 g/L dry weight of cells in fresh culture medium and keeping the subcultures under permanent stirring.

18. The method of claim 17, further comprising irradiating the suspension by a light emitting diode system.

19. The method of claim 17, wherein step (i) is performed first in a stirred vessel and second in a bubbling column type bioreactor.

20. A method for obtaining fiber-like structures from cultured meristematic cambial cells, the method comprising:
(i) suspending meristematic cells in a first culture medium comprising macronutrients, micronutrients, vitamins, antioxidants, and phytohormones;
(ii) taking and suspending the meristematic cells from step (i) again in a second culture medium comprising macronutrients, micronutrients, vitamins, antioxidants, and phytohormones;
(iii) stirring the suspension from step (ii) for a first induction period;
(iv) separating the cells from the second culture medium and adding a fresh amount of the second culture medium; and
(v) agitating the cells and the fresh amount of the second culture medium for a second induction period,
wherein the second culture medium comprises phytohormones different from the phytohormones of the first culture medium.

21. The method of claim 20, wherein the method starts in a stirred vessel and is subsequently performed in a bubbling column type bioreactor.

22. The method of claim 20, wherein the phytohormones of the first culture medium are selected from the group consisting of auxin, cytokines, gibberellins, ethylene, abscisic acid, and mixtures thereof.

23. The method of claim 20, wherein the phytohormones of the second culture medium are selected from the group consisting of auxins, gibberellins, cytokines, abscisic acid, and mixtures thereof.

24. The method of claim 20, further comprising performing a transcriptomic analysis of the meristematic cells and the fiber-like structures.
